(19) United States
(12) Patent Application Publication
(10) Pub. No.: US 2014/0251832 A1
(43) Pub. Date: Sep. 11, 2014

(54) Electrochemical Assay

(71) Applicant: The Secretary of State for Business Innovation & Skills of Her Majesty's Britanic Government, London, Greater London (GB)

(72) Inventor: Robert Andrew Porter, Sharnbrook (GB)

(21) Appl. No.: 14/352,642

(22) PCT Filed: Oct. 22, 2012
(86) PCT No.: PCT/GB2012/052615

§ 371 Date: Apr. 17, 2014

(30) Foreign Application Priority Data
Oct. 24, 2011 (GB) 11182993.8

(51) Int. Cl. G01N 27/327; G01N 33/487

(52) U.S. Cl. CPC G01N 27/3277; G01N 33/4875
USPC 205/780.5; 205/787; 205/782; 204/403.01

(57) ABSTRACT

An electrochemical assay uses paramagnetic particles (10) including a coating of electroactive material (12) in order to detect or measure an analyte (30) of interest. The analyte is brought within the vicinity of an electrode (42) along with the coated paramagnetic particles (10). Application of a potential converts the electroactive coating (12) on the paramagnetic particles (10) into ions that can be measured using, for example, anodic stripping voltammetry. The level of ions corresponds to the amount of analyte of interest in the sample.

The present application relates to an electrochemical assay, and in particular to a method for determining the presence or amount of an analyte in a sample.

Immunoassays are often used for the detection of a specific analyte within a sample. For example, antibodies to a particular biomarker, such as testosterone or cortisol, may be used to test levels of these substances in the saliva, blood or urine of an athlete.

WO 2005/121792 discloses the use of metal particles bound to a species of interest. The metal particles are dissolved, and then electrochemically measured to provide an indication of the presence or amount of the metal-labelled species. However, the methods require chemical oxidants in order to dissolve the metal particles. A problem with this is that the oxidant can interfere with the electrochemical profile of the scan by disrupting the baseline of the scan. The assay described in WO 2005/121792 relies on the formation of metal ions in order for the metal to be transferred to an electrode surface electroanalytically. This is problematic, in particular when analysing biological samples. Proteins and other molecules in a biological sample solution typically bind to the metal ions rendering them electrochemically inactive. The metal ions can also be rendered electrochemically inactive by chelation/coupling with the chemical oxidant. Accordingly, the sensitivity of the signal is reduced.

WO 2009/068862 discloses an assay that addresses the above problems. The assay takes advantage of a chemical release agent to release the metal label from an analyte of interest, the release agent and the metal label together forming a (typically negatively) charged species. An electrical potential is then applied to bring the charged species to an electrode. A positive potential is then applied to the charged species to form metal ions from the metal label. A quantitative determination procedure such as anodic stripping voltammetry (ASV) is then carried out to determine the presence or amount of the metal-labelled analyte. This assay requires the use of a chemical release agent.

The present invention seeks to overcome one or more of the above problems.

According to a first aspect of the present invention, there is provided a method for determining the presence or amount of an analyte in a sample, the method comprising the steps of: labelling the analyte with a paramagnetic particle coated with an electroactive material; applying a magnetic field to bring the labelled analyte to an electrode; applying a potential to the labelled analyte to form ions from the electroactive material; and carrying out a quantitative determination procedure to determine the presence or amount of the metal-labelled analyte.

The ions are formed very close to the electrode surface. There is therefore little opportunity for the ions to be deactivated before they are measured. It is therefore not necessary to form a complex between the ions and a chelating agent. Moreover, as an electrochemical potential is used to dissolve the electroactive material, it is not necessary to use a chemical oxidant and the problems associated with chemical oxidants are avoided. Furthermore, use of paramagnetic particles coated with an electroactive material enables measurement of the electroactive material at the electrode without the need to release the electroactive label from the analyte. This therefore avoids the need to use any chemical release agent.

In preferred embodiments, the labelled analyte binds to a binding moiety provided at a region of a sample carrier proximate the electrode.

Preferably, prior to carrying out the quantitative determination procedure, the magnetic field is reversed. As a result, only coated paramagnetic particles bound to the binding moieties via the analyte remain at the electrode, and thus only the electroactive material coating those particles is measured. This avoids the need for a wash step to remove unbound particles from the electrode.

According to a second aspect of the present invention there is provided a method for determining the presence or amount of an analyte in a sample, the method comprising the steps of: providing paramagnetic particles coated with an electroactive material and including a first binding moiety; providing a second binding moiety at a region of a sample carrier proximate an electrode; incubating a sample suspected of containing the analyte with the first and second binding moieties; applying a magnetic field to bring the paramagnetic particles to the electrode, applying a potential to the electroactive material to form ions, and carrying out a quantitative determination procedure to determine the presence or amount of the analyte.

If the target analyte is present, the electroactive paramagnetic particle can bind at the electrode surface via the binding moieties. Unbound particles can be moved away from the electrode by means of a magnetic field. The bound particles can then be measured by applying a potential to form ions and carrying out the quantitative determination procedure.

The term “incubating” is not intended to imply any particular process apart from allowing the sample to contact the binding moieties and for binding of the analyte with the appropriate binding moiety to occur.

In an embodiment, the first binding moiety and the second binding moiety are antibodies each specific to different epitopes of an analyte of interest. This may usefully be termed a “sandwich assay”. Such an assay may be useful for the detection and/or measurement of troponin I, a marker of myocardial cell death, human chorionic gonadotrophin in a pregnancy test, or thyroid stimulating hormone for monitoring thyroid function.

In certain embodiments, the first binding moiety or the second binding moiety may compete with the analyte for binding to the second binding moiety or the first binding moiety respectively. For example, the first binding moiety or the second binding moiety may be substantially identical to the analyte. This arrangement provides a competition assay (or hapten assay) and may be particularly useful where the analyte of interest only has a single epitope. A hapten assay might be particularly useful for testing athletes for levels of testosterone or cortisol, or for measuring levels of estradiol for measuring female fertility.

The first binding moiety and/or the second binding moiety may be antibodies.

In some embodiments, the analyte is an antibody and the first binding moiety and/or the second binding moiety may be a corresponding antigen. Where the analyte is an antibody, the first binding moiety or the second binding moiety may be a further antibody, for example, an anti-Ig antibody. Such assays may be termed “serological assays” and may generally be used to determine an antibody response to an immunisation or infection. The antigen may therefore be the infectious agent or a part thereof.

Of course, antibodies are merely examples of binding moieties. The binding moiety, or indeed the analyte, may be any suitable molecule such as a molecular imprinted polymer, DNA, RNA, single nucleotide polymorphisms, or a mimotope, for example.

The electroactive material may be a metal, in which case a positive potential is applied to form metal ions. In preferred embodiments, the metal may be gold, silver, copper or arsenic, for example. In other embodiments, the electroactive material may be non-metallic. For example, the electroactive material may be pyrol, thiophene or carbazol where the negative or positive redox process can be measured dependent on the material being used.

The quantitative determination procedure may be a voltammetric method, such as anodic stripping voltammetry (ASV).

Preferred features of the second aspect apply equally to the first aspect and vice versa.

According to a third aspect of the present invention there is provided a kit for use in the above-described methods, including a sample carrier for insertion into a detector unit, the sample carrier including a binding moiety bound thereto at a region of the sample carrier to be located proximate an electrode of the detector unit; and paramagnetic particles coated with an electroactive material and including a binding moiety.

The paramagnetic particles may in some embodiments be provided in the sample carrier.

Preferred embodiments will now be described by way of example only and with reference to the drawings in which:

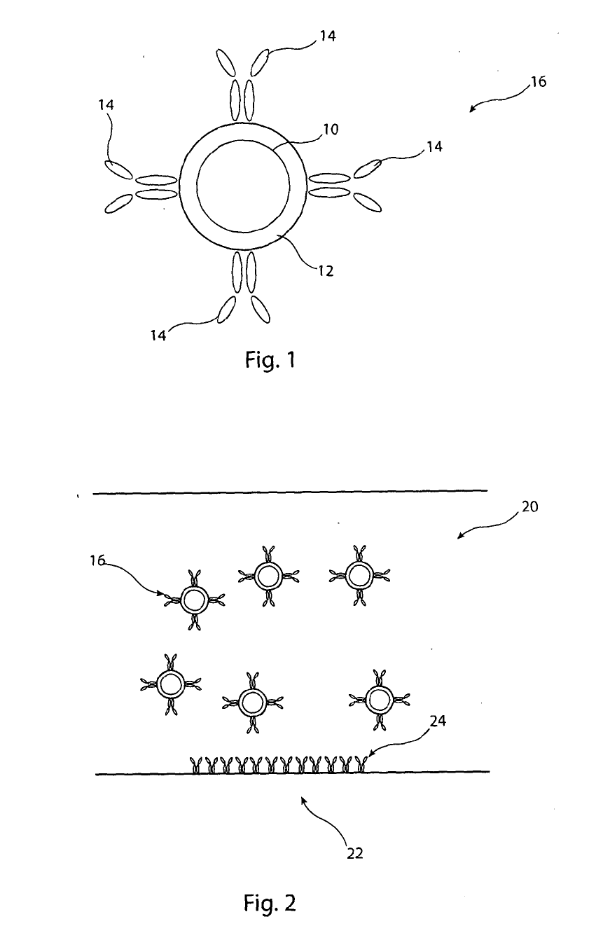

FIG. 1 is a schematic drawing of an embodiment of a paramagnetic particle;

FIG. 2 is a schematic drawing of the paramagnetic particles of FIG. 1 in a sample carrier;

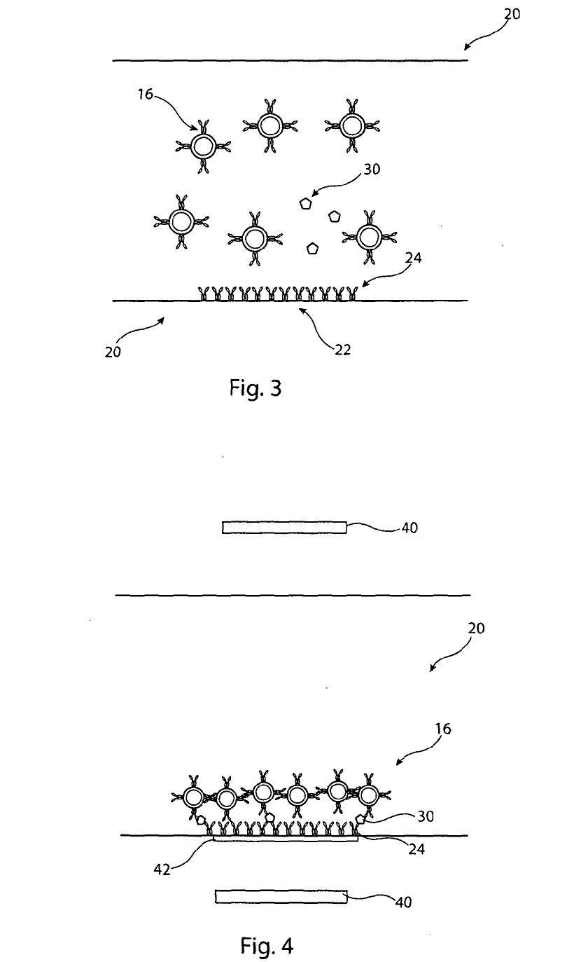

FIGS. 3 to 5 are schematic drawings illustrating the steps of an embodiment of a method;

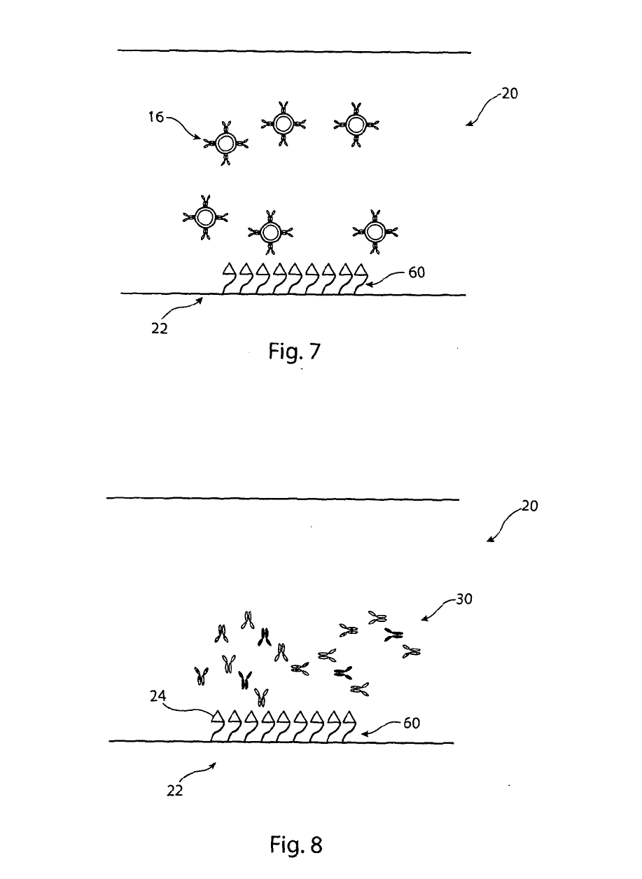

FIGS. 6 and 7 are schematic drawings illustrating further embodiments of a method; and

FIGS. 8 to 11 are schematic drawings illustrating a further embodiment of a method.

Referring firstly to FIG. 1, a paramagnetic particle 10 has been coated with silver 12 using a conventional method, such as chemical reductive deposition. On the surface of the silver-coated paramagnetic particle 10, there have been bound antibodies 14 specific to a first epitope of an analyte of interest. The entire particle is referred to hereinafter as a labelled paramagnetic conjugate 16.

FIG. 2 illustrates a sample carrier 20, a region 22 of the internal surface of which has been coated with an antibody 24 specific to a second epitope of the analyte of interest. The labelled paramagnetic conjugates 16 of FIG. 1 are also provided within the sample carrier 20, for example within an inert liquid carrier.

A sample to be tested for the analyte of interest, for example, a sample of saliva, blood, or urine, is introduced into the sample carrier 20. FIG. 3 illustrates the situation where antigen 30 is present in the sample. It can be seen that the antigen 30 will bind to both the antibody 14 provided on the paramagnetic particle and also to the second antibody 24 provided on the internal surface of the sample carrier 20.

After the necessary incubation period, which can be determined by the skilled person according to the particular antigen/antibody in question, a magnetic field is applied using a magnet 40 (which may be a solid magnet or an electromagnet, for example). The sample carrier 20 is, in preferred embodiments, inserted into a detector unit that includes the necessary magnet 40 and also an electrode 42 (see

FIG. 4). The magnetic field is introduced at the electrode 42, and thus attracts the paramagnetic particle conjugates 16 to the electrode 42, where a “sandwich” occurs if the antigen 30 is present.

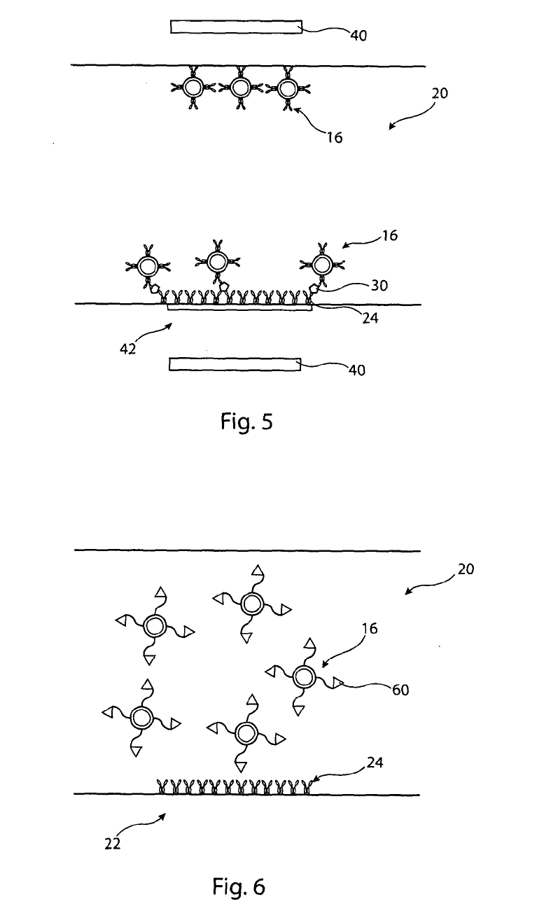

As illustrated in FIG. 5, subsequent reversal of the magnetic field causes unbound labelled paramagnetic conjugates 16 to be removed from the electrode 42 leaving only bound labelled paramagnetic conjugates 16 at the electrode 42.

A positive potential is then applied to the electrode 42 in order to convert electrochemically the silver metal 12 of the labelled paramagnetic conjugate 16 bound at the electrode 42 to silver metal ions. The resulting metal ions can then be measured using, for example, anodic stripping voltammetry (ASV). It will be appreciated that since only labelled paramagnetic conjugate 16 bound to antigen 30 is retained at the electrode, the measurement of the silver ions produced by oxidation is directly related to the quantity of antigen 30 of interest in the original sample. Any labelled paramagnetic conjugates 16 that do not bind to the second antibody 24 at the electrode 42 are removed from the electrode 42 (in this embodiment, by reversing the magnetic field). The silver coat 12 on these labelled paramagnetic conjugates 16 will therefore not affect the amount of silver ion generated by oxidation.

The above-described sandwich assay could be used, for example, as a test for myocardial cell death by measuring levels of troponin I, as a pregnancy test by measuring levels of human chorionic gonadotropin, or as a test for thyroid function by measuring levels of thyroid stimulating hormone.

There are several advantages to the above-described embodiment.

It is not necessary to include a washing step to remove unbound labelled paramagnetic conjugate 16 from the electrode 42 prior to oxidation of the silver 12. This is because unbound labelled paramagnetic conjugate 16 is removed from the electrode 42 by reversing the magnetic field.

ASV provides a direct measure of the quantity of silver ions produced, which in turn is directly proportional to the labelled paramagnetic conjugate 16 retained at the electrode 42, which in turn is directly proportional to the amount of antigen 30 present in the original sample.

FIG. 6 illustrates a hapten assay. This embodiment is similar to that of FIGS. 2 to 5, but instead of being coated with an antibody, the labelled paramagnetic conjugates 16 are bound to a hapten 60, which is a conjugated version of the antigen of interest, and competes with the antigen for binding to the antibodies 24.

In this case, any antigen in the sample binds the antibody 24, and prevents binding of the hapten-labelled paramagnetic conjugates 16. In this case, therefore, the level of metal ions measured by ASV would be inversely proportional to the amount of antigen in the sample.

FIG. 7 illustrates a method very similar to that illustrated in FIG. 6. However, as a modification, hapten 60 is provided at the region 22 of the sample carrier 20 that is to be located near the electrode, and antibody 14 is provided to form the labelled paramagnetic conjugates 16. Any antigen in the sample will bind to the antibodies 14, thereby preventing binding of the labelled paramagnetic conjugate 16 to the hapten 60. Again, the level of metal ions measured by ASV is inversely proportional to the amount of antigen in the original sample.

A hapten assay would be particularly useful for antigen having only a single epitope, and therefore could be used to test athletes for testosterone or cortisol, for example.

In an further embodiment, a serological assay is provided. In this case, the analyte is an antibody, and the assay may be used to measure immunological response to an infection or an immunisation.

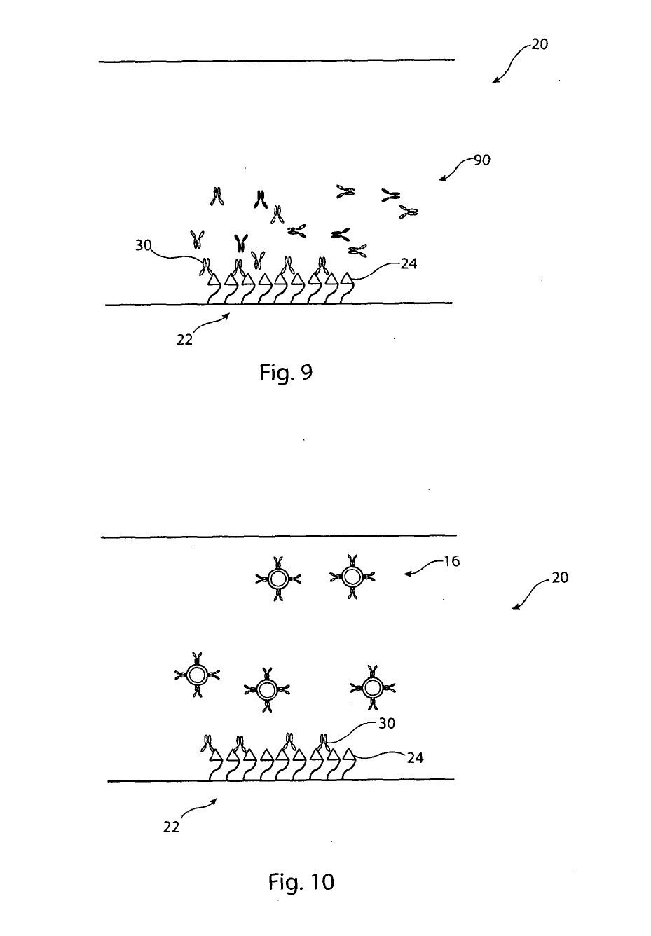

As shown in FIGS. 8 and 9, the analyte 30, which is a specific antibody, is able to bind to an antigen 24 provided at a region 22 of the sample carrier 20 that is to be located next to an electrode. After allowing binding between the antibody 30 and the antigen 24, a washing step is carried out in order to remove unbound antibody 90.

Labelled paramagnetic conjugate 16 is then introduced into the sample carrier 20 (see FIG. 10). The labelled paramagnetic conjugate 16, in this example, is conjugated to a secondary antibody 14 that binds to the antibody 30 of interest. The secondary antibody 14 may therefore be an anti-Ig antibody. In a modification, the labelled paramagnetic conjugates 16 used in a serological assay may be provided with the antigen 24.

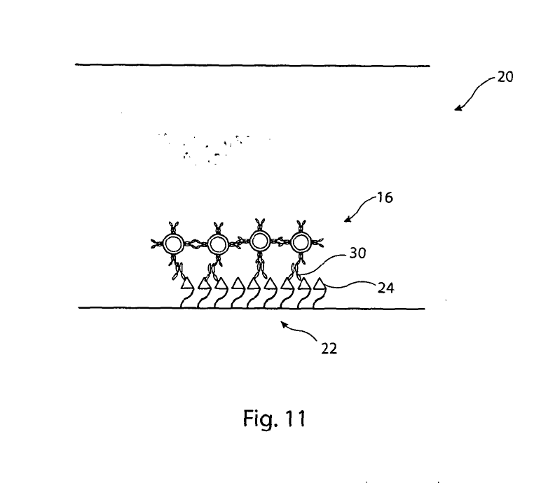

As shown in FIG. 11, labelled the paramagnetic conjugate 16 binds to antigen 24 at the region 22 of the sample carrier 20 to be located next to an electrode, and can be measured by oxidation followed by ASV as described above.

It is envisaged that the sample and the labelled paramagnetic conjugate 16 be mixed together within a sample carrier 20, which is then introduced into a detector unit that contains the magnets 40 and the electrode 42, and which is further operable to interpret and display the results of the assay. As such, each sample carrier 20 can be preloaded with appropriate binding moieties (for example, antibodies and, in some embodiments, the labelled paramagnetic conjugates 16 themselves) for a particular analyte of interest. Each sample carrier 20 may thus be intended for a single use, with the more expensive electronics being found within a reusable detector unit. The device disclosed in the applicant's earlier PCT application published as WO 2010/004244 could be readily adapted for performing the methods disclosed in the present application.

The skilled person would appreciate that the above description provides exemplary methods, and that many modifications may be made thereto.

The illustrated arrangements of antibodies, antigen and hapten are merely exemplary. The skilled person would know how to adapt the methods described in order to test for other types of analyte.

Silver is only one example of suitable electroactive material. Whilst it may be preferred because it can easily be oxidised electrochemically to form silver ions, other electroactive materials may also be suitable. In particular, metals such as gold, copper or arsenic, and non-metals such as pyrol, thiophene or carbazol may be suitable.

In a modification, two different analytes within the same sample can be detected by using labelled paramagnetic conjugates coated with two different electroactive materials, for example, with gold or with silver. For example, gold-coated paramagnetic particle may be attached to a first binding moiety that recognises a first analyte and silver-coated paramagnetic particles may be attached to another binding moiety that recognises a second analyte. Gold ions and silver ions give different distinguishable peaks when measured on the same electrode by ASV.

The above-described embodiments provide a simple assay for an analyte of interest, which can be carried out in a single chamber to allow for separation of unbound labelled paramagnetic conjugate from labelled paramagnetic conjugate that has bound to target analyte. Incubation, separation and measurement can thus all take place within the same chamber allowing for simplified mechanics and electronics. Furthermore, other than the standard chemicals used in any controlled bio-assay (such as sample and pH buffers) no additional chemical reagent is required, in contrast to the oxidant and release agent required in the prior art.

The disclosures in United Kingdom patent application GB 1118293.8 and in the abstract accompanying this application are incorporated herein by reference.

1-32. (canceled)

33. A method for determining the presence or amount of an analyte in a sample, the method comprising the steps of:
providing paramagnetic particles coated with an electroactive material and including a first binding moiety;
providing a second binding moiety within a sample carrier;
incubating a sample suspected of containing the analyte with the first and second binding moieties;
applying a magnetic field to bring the paramagnetic particles to an electrode;
applying a potential to the electroactive material to form ions; and
carrying out a quantitative determination procedure to determine the presence or amount of the analyte

34. The method of claim 33, wherein the first binding moiety and/or the second binding moiety are an antibody.

35. The method of claim 34, wherein the first binding moiety and the second binding moiety are antibodies each specific to different epitopes of the analyte.

36. The method of claim 35, wherein the analyte is troponin I, human chorionic gonadotrophin, or thyroid stimulating hormone.

37. The method of claim 33, wherein the first binding moiety or the second binding moiety competes with the analyte for binding to the second binding moiety or the first binding moiety respectively.

38. The method of claim 37, wherein the first binding moiety or the second binding moiety is substantially identical to the analyte.

39. The method of claim 37, wherein the analyte only has a single epitope.

40. The method of claim 37, wherein the analyte is testosterone, cortisol, or estradiol.

41. The method of claim 33, wherein the analyte is an antibody and the first binding moiety and/or the second binding moiety is a corresponding antigen.

42. The method of claim 41, wherein the first binding moiety or the second binding moiety is a further antibody.

43. The method of claim 42, wherein the first binding moiety and/or the second binding moiety is an anti-Ig antibody.

44. The method of claim 41, wherein the method is for determining an antibody response to an immunisation or infection.

45. The method of claim 44, wherein the antigen for the analyte antibody is an infectious agent or a part thereof.

46. The method of claim 33, wherein the analyte is a molecular imprinted polymer, DNA, RNA, a single nucleotide polymorphism, or a mimotope.

47. The method of claim 33, wherein the first binding moiety and/or the second binding moiety is a molecular imprinted polymer, DNA, RNA, a single nucleotide polymorphism, and/or a mimotope.

48. The method of claim 33, wherein prior to carrying out the quantitative determination procedure, the magnetic field is reversed.

49. The method of claim 33, wherein the electroactive material is a metal, and a positive potential is applied to form metal ions.

50. The method of claim 49, wherein the metal is gold, silver, copper or arsenic.

51. The method of claim 33, wherein the electroactive material is non-metallic.

52. The method of claim 51, wherein the electroactive material is pyrol, thiophene or carbazol.

53. The method of claim 33, wherein the quantitative determination procedure is a voltammetric method.

54. The method of claim 53, wherein the quantitative determination procedure is anodic stripping voltammetry (ASV).

55. A kit for use in the method of claim 33, including a sample carrier for insertion into a detector unit, the sample carrier including a binding moiety bound thereto at a region of the sample carrier to be located proximate an electrode of the detector unit; and paramagnetic particles coated with an electroactive material and including a binding moiety.

56. A kit as claimed in claim 55, wherein the paramagnetic particles are provided in the sample carrier.
